(19) United States
(12) Patent Application Publication
(10) Pub. No.: US 2012/0310284 A1
(43) Pub. Date: Dec. 6, 2012

(54) POLYAXIAL PEDICLE SCREW

(75) Inventor: James R. Gerchow, Carson City, NV (US)

(73) Assignee: Royal Oak Industries, Bloomfield Hill, MI (US)

(21) Appl. No.: 13/152,674

(22) Filed: Jun. 3, 2011

(51) Int. Cl. A61B 17/70; B23P 11/00; A61B 17/86

(52) U.S. Cl. 606/264; 606/305; 29/428

(57) ABSTRACT

A spinal stabilization assembly is provided and includes a cup, fastener and keeper portions surrounding the head of the fastener. Further, methods of use of a spinal stabilization assembly are also provided. The keeper portions are located within the cup and at least partially surround the head of the fastener.

### BACKGROUND OF THE INVENTION

1. Field of the Invention

The embodiments disclosed herein relate to assemblies used to stabilize spinal segments or other bone structures.

2. Description of the Related Art

Spinal stabilization assemblies are used in order to secure adjacent sections of the vertebral column. Stabilization procedures are performed in spinal fusion surgeries, where an intervertebral disc is wholly or partially removed and the adjacent vertebra are fused with bone to combine adjacent segments of the vertebral column, creating a larger singular bone structure. This type of treatment can be used to treat spinal deformity, injury or disease.

Pedicle screws have been widely used in such treatments for over forty years. Pedicle screws serve as anchor points in adjacent or nearby vertebrae that can then be connected with a rod or the like to stabilize the spinal segment while the adjacent vertebra fuse. Such spinal assemblies largely use polyaxial screw systems to accomplish vertebral connection. Polyaxial screws generally comprise a spherical head enclosed in a housing that is able to pivot at any angle relative to the polyaxial screw. Polyaxial screw systems are described in U.S. Pat. No. 5,207,678 to Harms et al and U.S. Pat. No. 5,690,630 to Errico et al.

Because the polyaxial pedicle screw serves as the anchor upon which the connecting components rely for their orientation, it is important that the pedicle screw is secured in a proper position with respect to the other components of the assembly. This allows physicians to create a secure and dependable fused spinal segment.

### SUMMARY OF THE INVENTION

According to one aspect of the invention, a spinal stabilization assembly is provided. The spinal stabilization assembly may comprise a cup, a fastener, and keeper portions. The cup comprises an aperture at its lower end and opposing rod channels. The fastener comprises a head and body and may extend through the lower end of the cup. The keeper portions have an outer surface, at least some of which is in contact with an internal surface of the cup. The keepers also have an inner surface shaped to mate with and at least partially surround the head of the fastener.

In certain aspects of the invention, the keepers may have a groove configured to receive a ring. The keepers may alternatively have a groove configured to receive a flange encircling the fastener head. They may also comprise a material softer than the material comprising the head of the fastener.

In certain aspects of the invention, the fastener may comprise a tapered cylindrical head. The body of the fastener may taper outwardly in a portion of the body proximate to its head. The fastener may comprise a lip proximate to its head. The fastener may be a pedicle screw.

In some aspects of the invention, the spinal stabilization assembly may also comprise a saddle which is located above the fastener. The saddle may be configured to exert pressure on the plurality of keeper portions. The assembly may comprise a compression member attached at the upper aperture of the cup. The compression member may comprise a threaded fastener which mates with a threaded portion of the cup. A rod may extend through the side channels of the cup and be located between the compression member and the head of the fastener.

In another aspect of the invention, a method for assembling a spinal stabilization assembly is provided and comprises inserting a fastener through an aperture of a cup, inserting one or more keeper portions through an aperture in a side wall of the cup, and arranging the keeper portions around a head of the fastener such that they at least partially surround the head of the fastener and have their outer surface in contact with an internal surface of the cup. The fastener may comprise a pedicle screw.

The method may further comprise inserting a saddle through the aperture in a side wall of the cup and placing the saddle so that its bottom portion mates with the outer surface of the keeper portions. The method may further comprise compressing the saddle and fixing the saddle in its compressed state. The method may also comprise placing a rod on top of the saddle, the rod extending through one or more side channels of the cup. A compression member may be inserted through an upper aperture of the cup and coupled to the cup so that it exerts pressure on the fastener.

According to yet another aspect of the invention, a spinal stabilization assembly is provided. The assembly comprises a cup with an aperture at its lower end and opposed rod channels. The assembly further comprises a fastener comprising a head and a body and extending through an aperture at the lower end of the cup. The assembly may also comprise means for clamping the head of the fastener into the cup, where the means is positioned between the cup and the head of the fastener. This means may further comprise means for biasing the clamping means outward towards the cup.

In another aspect of the invention, a spinal stabilization assembly is provided. The assembly comprises a cup with an aperture at its lower end and opposed rod channels. It further comprises a fastener with a head and a body extending through an aperture at the lower end of the cup. The assembly also comprises one or more keeper portions that have an outer surface and an inner surface. The inner surface is shaped to mate with and partially surrounds the head of the fastener. At least some of the outer surface of the keeper portions is in contact with an internal surface of the cup. The keeper portions may comprise a material that is softer than the material comprising the fastener or the cup. The keeper portions may comprise a material with a yield strength of less than or equal to about 50 ksi. One or both of the fastener and the cup may comprise a material with a yield strength of greater than or equal to about 100 ksi.

### BRIEF DESCRIPTION OF THE DRAWINGS

FIG. 1A is a depiction of a spinal stabilization assembly according to one embodiment.

FIG. 1B is another depiction of a spinal stabilization assembly according to one embodiment.

FIG. 2 is an illustration of a fastener according to one embodiment.

FIG. 3A is a depiction of keeper portions, a ring and a fastener according to one embodiment.

FIG. 3B is an illustration of the keeper portions of FIG. 3A oriented together around the fastener head.

FIG. 4A is a depiction of a cup according to one embodiment.

FIG. 4B is a view of the rotated cup.

FIG. 4C is a cross-sectional view of a cup according to one embodiment.

FIG. 4D is another cross-sectional view of the cup.

FIG. 5A is an illustration of a saddle according to one embodiment.

FIG. 5B is a cross sectional view of the saddle of FIG. 5A.

FIG. 6 is a depiction of a compression member according to one embodiment.

FIG. 7 is an illustration of another embodiment of a spinal stabilization assembly.

FIG. 8 is a depiction of another embodiment of a fastener.

FIG. 9A is an illustration of another embodiment of keeper portions and a fastener.

FIG. 9B is a depiction of the keeper portions of FIG. 13A oriented together around a fastener head.

FIG. 10A is a depiction of a spinal stabilization assembly according to one embodiment.

FIG. 10B is another depiction of a spinal stabilization assembly according to one embodiment.

FIG. 11A is a top perspective view of a saddle according to one embodiment.

FIG. 11B is a bottom perspective view of the saddle of FIG. 11A.

FIG. 11C is a cross section of the saddle of FIGS. 11A and 11B along lines 11C of FIG. 11A.

FIG. 11D is a cross section of the saddle of FIGS. 11A and 11B along lines 11D of FIG. 11A.

FIG. 12A is a depiction of a cup according to one embodiment.

FIG. 12B is a view of the rotated cup.

FIG. 12C is a cross-sectional view of a cup according to one embodiment.

FIG. 12D is another cross-sectional view of the cup.

FIG. 13A is an illustration of a fastener according to one embodiment.

FIG. 13B is another illustration of a fastener according to one embodiment.

FIG. 14A is depiction of an embodiment of keeper portions and a fastener.

FIG. 14B is a depiction of the keeper portions of FIG. 14A oriented together around a fastener head.

### DETAILED DESCRIPTION OF THE PREFERRED EMBODIMENT

The embodiments disclosed herein relate to assemblies and methods of use for spinal stabilization.

Embodiments will now be described with reference to the accompanying Figures, wherein like numerals refer to like elements throughout. The terminology used in the description presented herein is not intended to be interpreted in any limited or restrictive manner, simply because it is being utilized in conjunction with a detailed description of certain specific embodiments disclosed herein. Furthermore, embodiments disclosed herein may include several novel features, no single one of which is solely responsible for its desirable attributes or which is essential to the embodiments herein described.

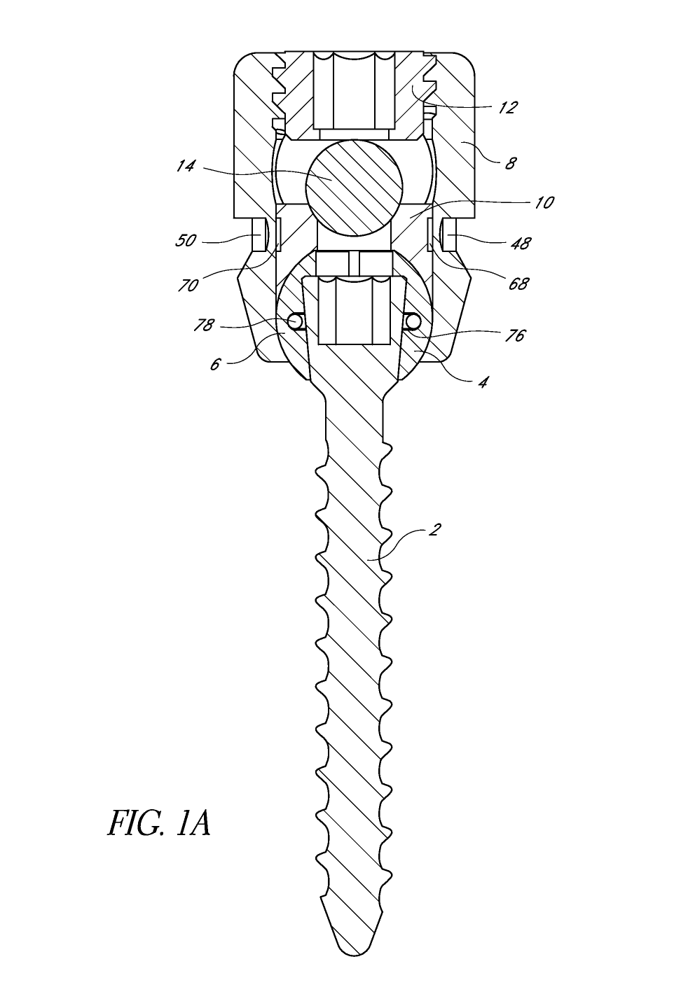

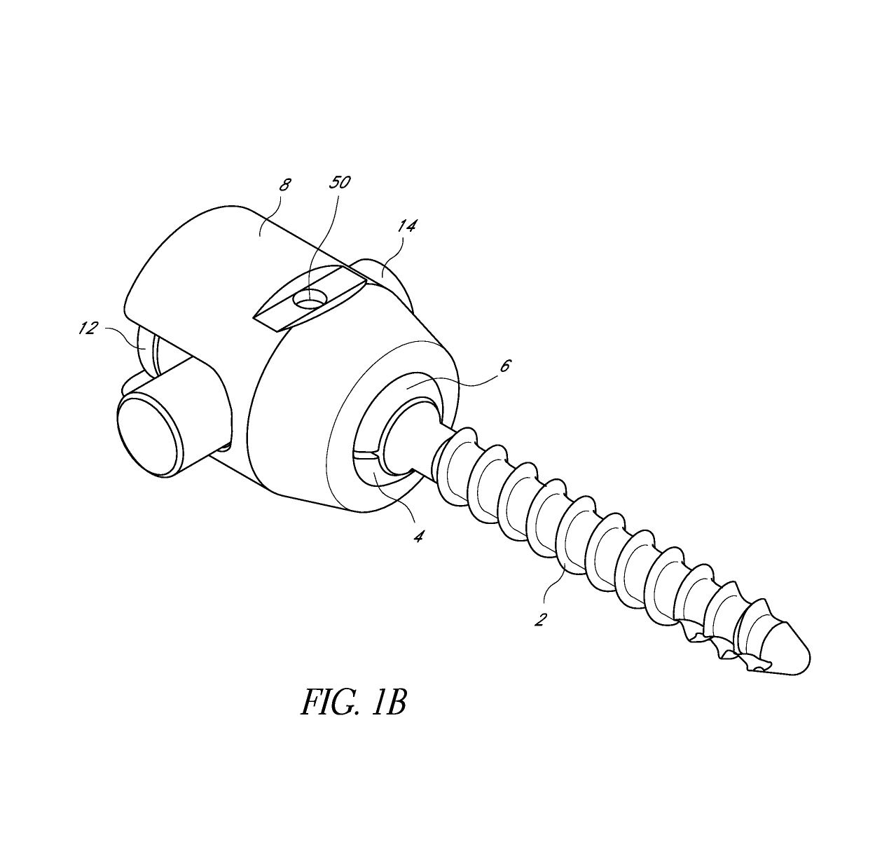

Referring now to FIGS. 1A and 1B, a spinal stabilization assembly according to one embodiment is shown. The assembly comprises several components which will be described in more detail below. FIG. 1A depicts a cross-sectional view of an assembled device. A fastener 2 extends through a bottom aperture of a cup 8. In some embodiments, the fastener comprises an elongate threaded shank and a head. In some embodiments, the assembly includes two keeper portions 4, 6 surrounding the head. The keeper portions 4, 6 comprise a significant novel feature of some embodiments and will be discussed in greater detail below. A saddle 10 may be located above the fastener 2 and inside the cup 8. A rod 14 sits inside the cup 8 and is located above the saddle 10. A threaded compression member 12 is screwed onto the rod 14, compressing the components below the compression member 12 within the cup 8. FIG. 1B depicts an assembled device in perspective view. The compression member 12 is visible through a side channel 38 of the cup 8. The rod 14 is shown below the compression member 12. The bottom portion of keeper portions 4, 6 are visible through an aperture at the bottom of the cup 8. The fastener 2 is shown extending through the bottom of the cup 8. The described assembly is a polyaxial screw system and so, due to the ball and socket type design of the fastener 2 in the cup 8, the cup 8 can pivot at any angle about the fastener 2.

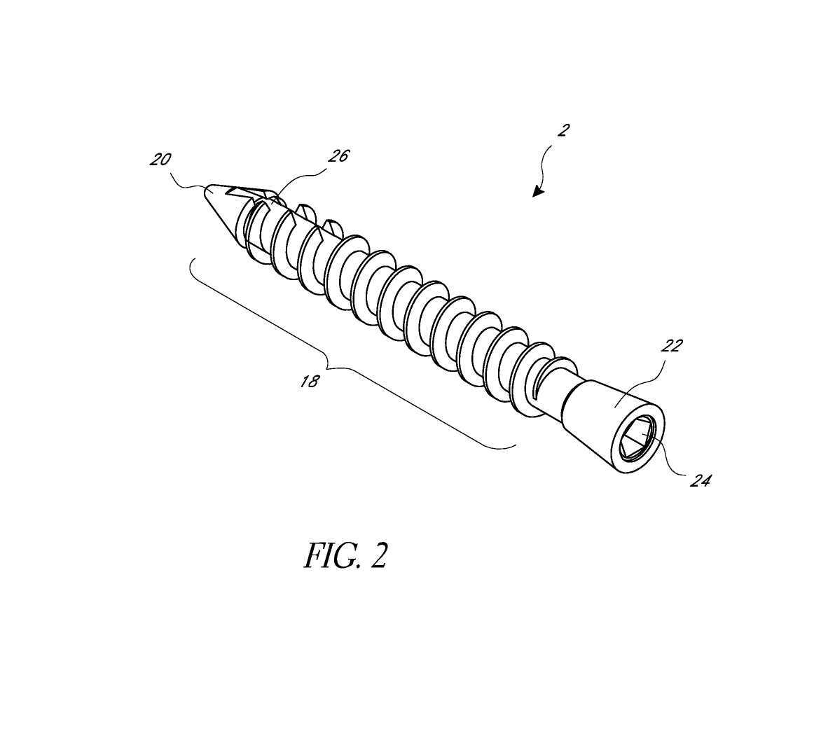

Moving to FIG. 2, an embodiment of a fastener is depicted. In some embodiments, the fastener may comprise an elongate screw containing a threaded shank 18 and a head 22. The screw head may be frustoconically shaped such that it comprises a tapered cylinder in which the circumference of the top of the screw head is greater than that of the bottom. Other shapes are also possible (e.g., cylindrical, spherical, etc.). The head of the screw may comprise a drive coupler 24 which can engage a tool used to manipulate the fastener in order to threadably engage the shank of the fastener with the vertebral body. In some embodiments, the drive coupler 24 comprises a recess whose shape may be a variety of forms. For example, the recess may be hexagonal in order to engage an Allen-style driver. However, other shapes and methods of coupling are also possible.

The threaded shank 18 may include a tip 20 suitable for piercing bone such as a pedicle. The threaded shank 18 may also comprise a groove 26 located near the tip 20 of the shank. This groove 26 can create a self-tapping screw as the edges of the groove are capable of piercing the bone to create a thread pattern. The groove 26 may be rounded, angular, or square. Other configurations are also possible. In some embodiments, the tip of the fastener 2 is not capable of piercing the bone. In such embodiments, a hole may be drilled and the fastener 2 may be inserted into the hole. While the fastener 2 is depicted in the form of an elongate screw in FIG. 2, other embodiments are possible, such as a hook configuration.

It will be appreciated that the width of the fastener 2 may vary, depending on the dimensions of the other components of the assembly (e.g., the keepers, the bottom aperture of the cup). It will also be appreciated that the length of the fastener 2 may vary. In some embodiments, the dimensions of the fastener 2 may be selected based on properties of the vertebral body in which to fastener 2 is to be inserted (e.g., brittleness, density, strength, etc.).

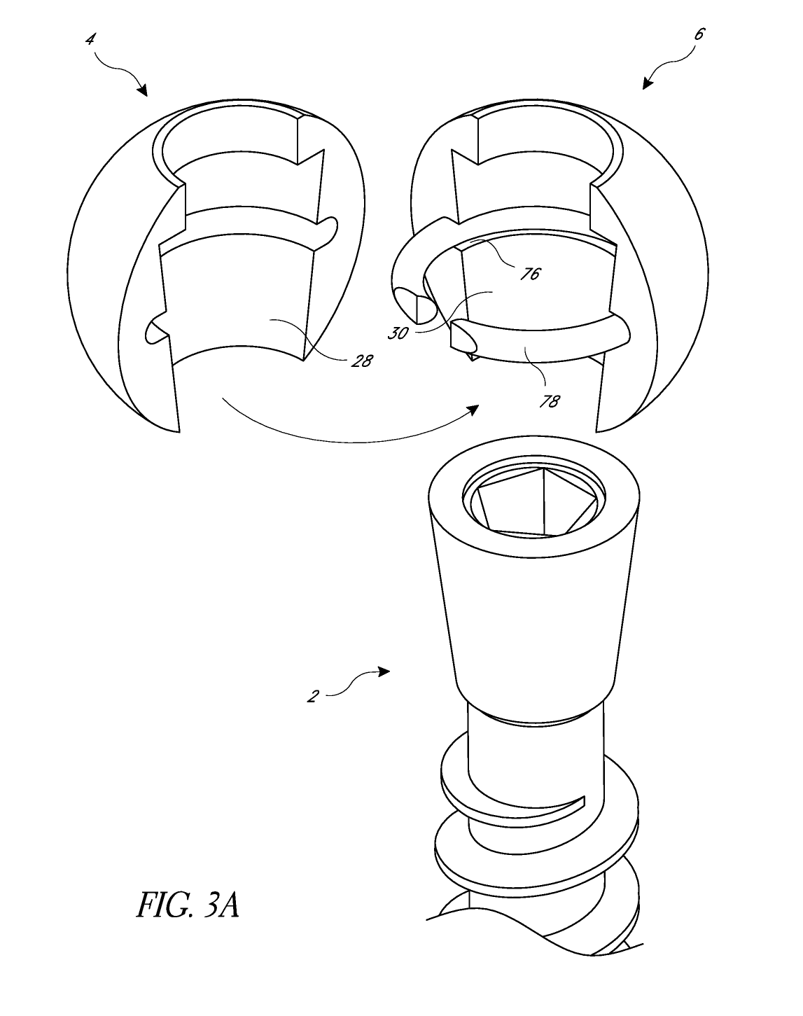

Referring now to FIGS. 3A and 3B, embodiments of the keeper portions 4, 6 are depicted. In some embodiments, the outer portion of the keepers 4, 6 are rounded and form a generally round or spherical shape when surrounding a fastener head, as depicted in FIG. 3B. The inside surfaces 28, 30 of the keepers 4, 6 are shaped to mate with the head of the fastener 2. When placed around a fastener head, the keepers 4, 6 form an upper aperture 31 which allows for access to the drive coupler 24 of the fastener 2.

In some embodiments, the keepers 4, 6 comprise a groove 76 as shown in FIG. 3A. A retention ring 78 can sit in this groove 76 between the keepers 4, 6, and the fastener 2. The ring 78 may comprise a metallic wire, such as a titanium wire or the like. The retention ring 78 may advantageously create an outward force on the keepers 4, 6, which in turn causes the keepers 4, 6, to exert an outward force on the cup 8. This force can cause increased friction between the keepers 4, 6, and the cup 8 before the assembly is locked by the surgeon. This extra friction can allow the surgeon to orient the cup 8 prior to placing the rod 14, and this position will be maintained by the friction between the keepers 4, 6 and the cup 8 as the rod 14 is manipulated into position in the rod receiving channels 38, 40 in the cup 8.

In some embodiments, the keepers may comprise a softer metal or material than that comprising the fastener. A softer material will deform when the assembly is locked by the compression members. The keepers will deform around and squeeze the fastener head to produce strong coupling. This deformation allows the fastener/keeper combination to be gripped more effectively than a monolithic fastener head. This more effective grip can be advantageous in securely maintaining a desired orientation for long periods after surgical implantation.

In some embodiments, the keepers may comprise a material with a yield strength of less than or equal to about 50 ksi. For example, Grade 2 unalloyed titanium (e.g. Ti-CP Gr2) has been found suitable. This material has a yield strength of about 40 ksi.

The other components, like the fastener and cup, may comprise a material with a yield strength of greater than or equal to about 100 ksi. In some embodiments, these components comprise Grade 5 titanium alloy (e.g. Ti-6Al-4V). Ti-6Al-4V is a titanium alloy containing aluminum and vanadium. This material has a yield strength of about 160 ksi, much higher than that of Ti-CP Gr2.

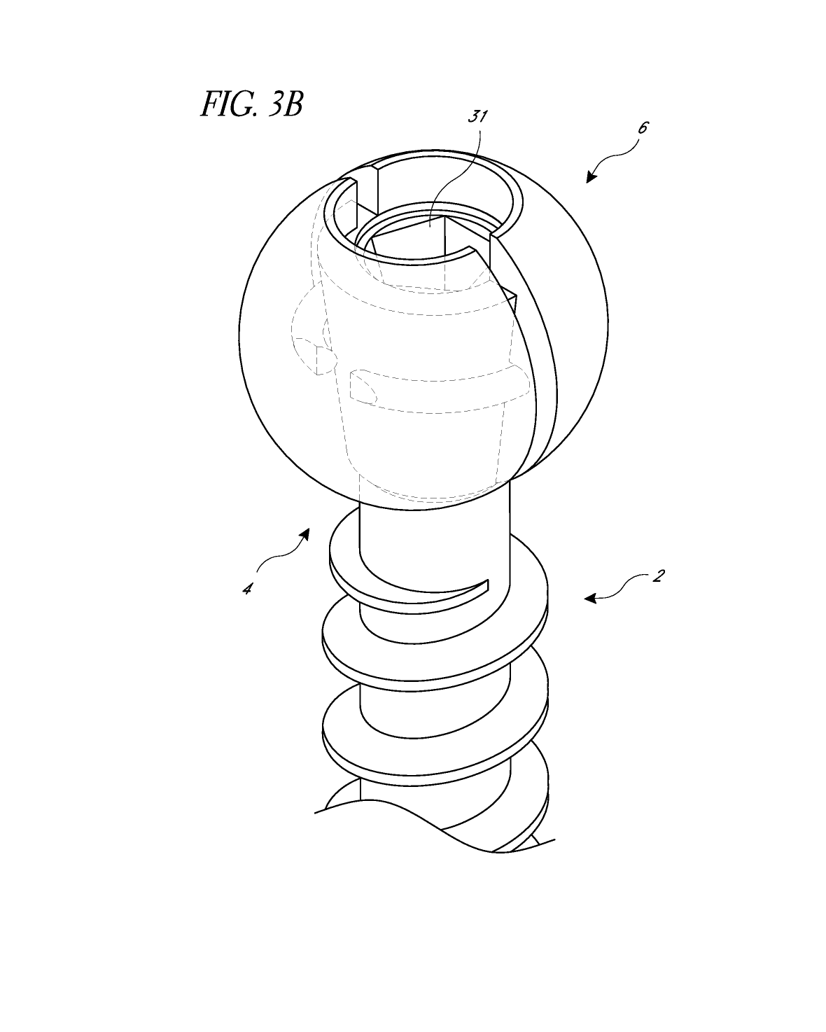

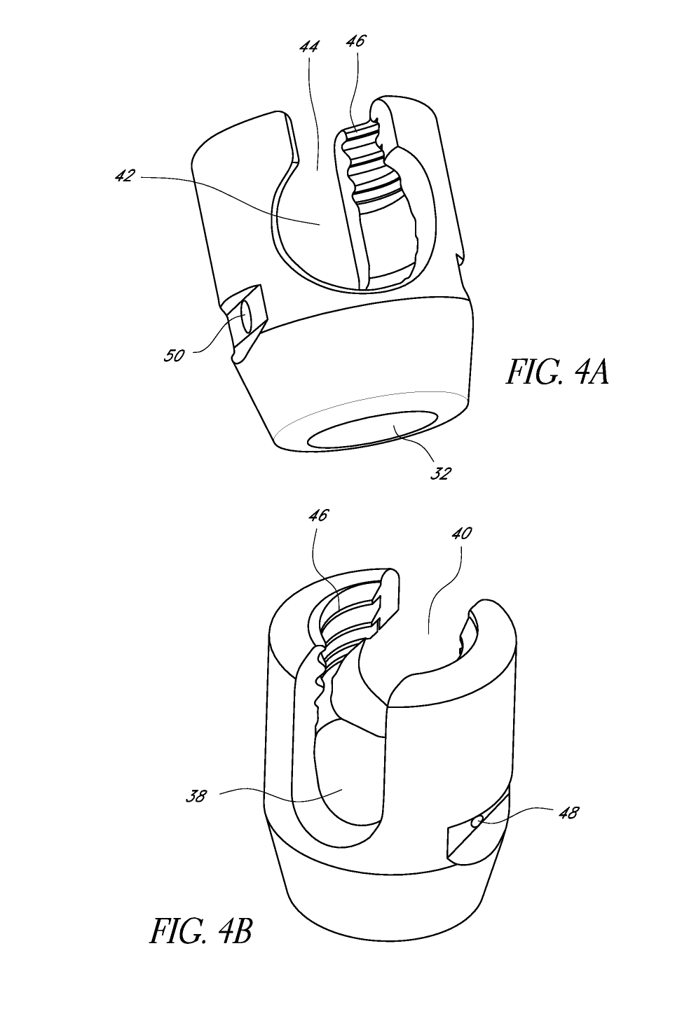

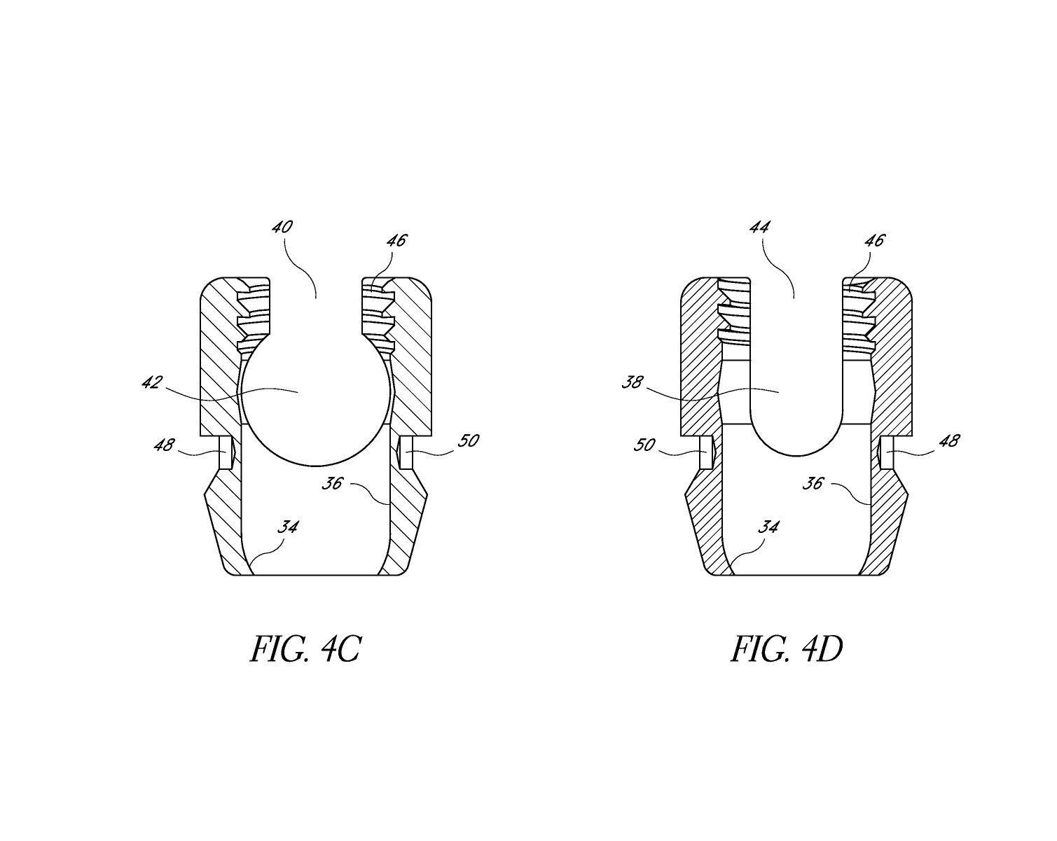

Turning to FIGS. 4A-4D, different perspectives of an embodiment of a cup 8 are shown. FIGS. 4A-4D illustrate an upper aperture 44 of the cup 8 (only labeled in FIGS. 4A & 4D for the purpose of clarity). This aperture 44 may be used for receiving a compression member 12. In some embodiments, there are threads 46 located inside the cup beneath the upper aperture 44 that can be used to engage a threaded compression member 12. Alternatively, the threads could be located on the outside of the cup to engage a compression member that contains threads along an inner surface. Other methods for coupling the compression member to the cup are also possible.

The sidewalls of the cup 8 may contain channels (e.g., U-shaped channels) 38, 40, shown in FIGS. 4B-4D, which can be used to receive a rod 14. FIGS. 4A and 4C show an opening 42 in a sidewall of the cup 8 that can be used for inserting the keeper and the saddle components. The opening 42 may be circular, rectangular or ovular. Other shapes are also possible.

As depicted in FIGS. 4A-4D, the sidewalls of the cup may also contain recesses 48, 50 where the thickness of the sidewalls is much lower than the thickness elsewhere on the cup 8. This decreased thickness enables the punching or dimpling of the sidewall in recesses 48, 50 to hold the saddle 10 in place within the cup 8. FIGS. 4C and 4D show the cylindrical inner surface 36 of the cup 8 where the saddle 10 is received.

As shown in FIGS. 4A, 4C, and 4D, the cup 8 contains a bottom aperture 32. The shank of the fastener may extend through this aperture 32 as shown in FIGS. 1A and 1B. The inner bottom surface 34 of the cup may have a rounded or spherical curvature, as depicted in FIGS. 4C and 4D. This curvature allows the generally spherically shaped keepers to seat the fastener at a number of different angles while still maintaining good contact with the cup 8.

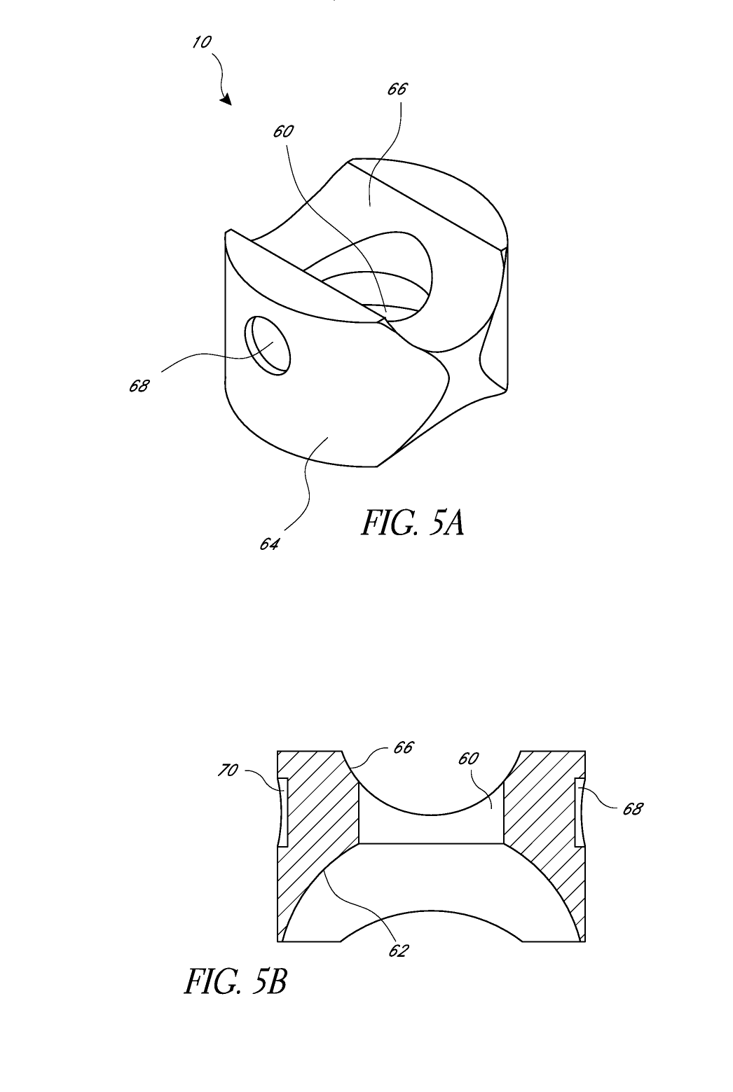

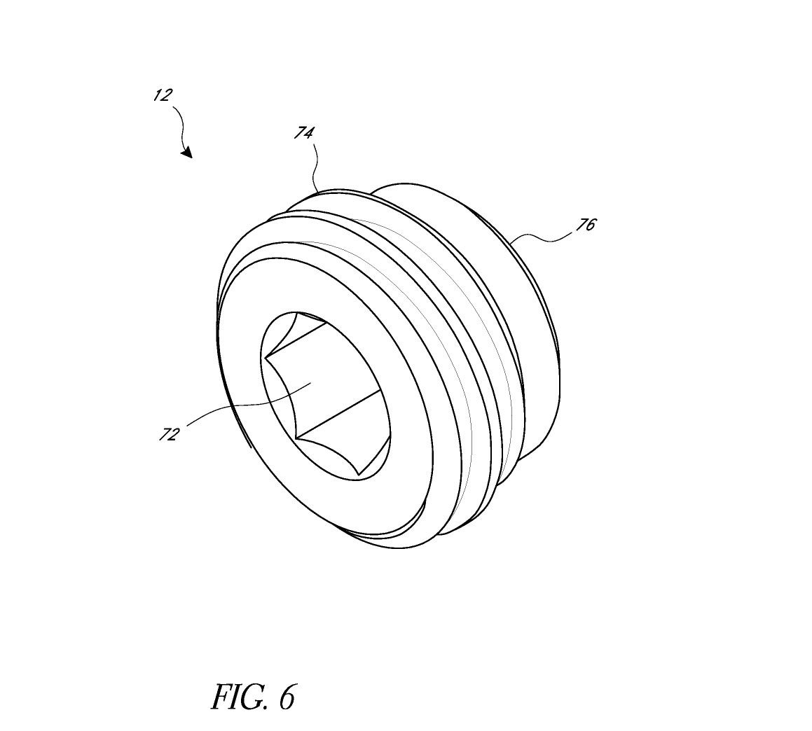

Moving to FIGS. 5A and 5B, an embodiment of a saddle 10 is depicted. The saddle 10 may contain a center bore 60 to allow access to the drive coupler of a fastener. In some embodiments, the outer surface 64 of the saddle forms a generally cylindrical shape which fits inside the similarly shaped portion 36 of the cup 8. The upper surface of the saddle may include a surface 66 shaped to mate with a support rod 14. In some embodiments, the sidewalls of the saddle include recesses 68, 70. These recesses 68, 70 correspond to recesses 48, 50 in the cup 8. The cup 8 may be punched or dimpled in recesses 48, 50 to make the sidewall deform into recesses 68, 70, holding the saddle 10 in place within the cup 8. The underside 62 of the saddle, shown in FIG. 5B, may form a rounded shape which can mate with the top of the keepers 4, 6.

FIG. 6 illustrates an embodiment of a compression member 12. The top surface of the compression member 12 may include a drive coupler 72. For example, in some embodiments, the drive coupler may engage an Allen-style wrench. However, other shapes and methods of coupling are also possible.

The underside 76 of the compression member may comprise a flat surface which interacts with the upper surface of the support rod 14. In other embodiments, the underside of the compression member may be round or contoured while still allowing interaction with the rod 14. Other topographies for the underside are also possible.

In certain embodiments, the compression member 12 comprises a threaded outer surface 74 which can be used to engage a threaded inner surface of the cup 8. In other embodiments, the inner surface of the compression member may be threaded and engage with an outer threaded surface of the cup. Other variations on the means for attaching the compression member 12 to the cup 8 are also possible.

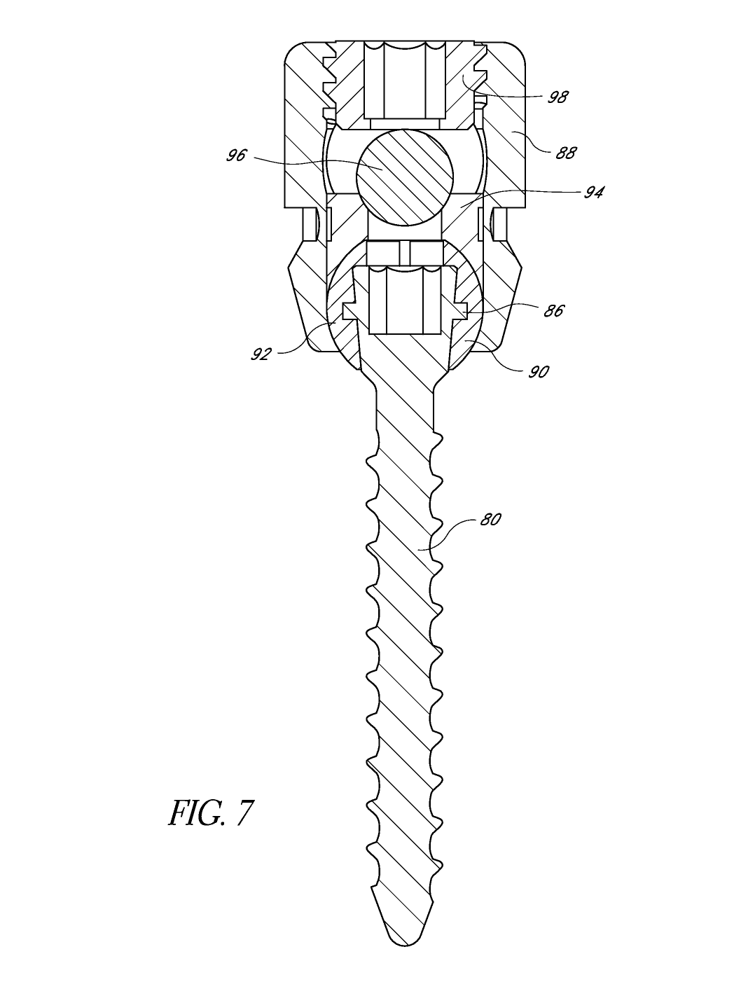

Referring now to FIG. 7, another embodiment of a spinal stabilization assembly is depicted. Unless otherwise described, the components of the assembly in FIG. 7 may be similar to the components described with respect to the assembly shown in FIGS. 1A and 1B. The assembly shown in FIG. 7 comprises a fastener 80 extending through a bottom aperture of a cup 88. The fastener 80 comprises an elongated threaded shank 82 and a head 84. In some embodiments, the fastener comprises a flange 86 encircling its head. The assembly may also include two keeper portions 90, 92 surrounding the head 84 of the fastener 80. In some embodiments, a saddle 94 may be positioned above the keepers 90, 92. The assembly may further comprise a rod 96 which sits inside the cup 88, above the saddle 94. A threaded compression member 98 may be screwed onto the rod, compressing components of the assembly within the cup 88.

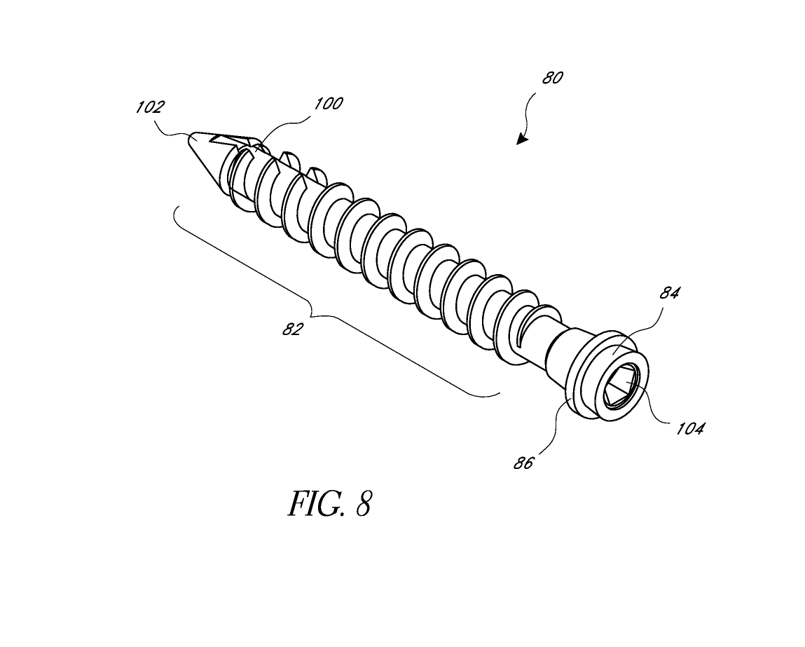

Turning to FIG. 8, an embodiment of the fastener 80 is depicted. The fastener may comprise an elongate screw comprising a threaded shank 82 and a head 84. The threaded shank 82 may comprise a groove 100 located near the tip 102 of the shank. As described above with reference to FIG. 2, this groove 100 may be capable of tapping the bone as the fastener 80 is screwed into the bone. In some embodiments, the fastener head 84 is frustoconically shaped. The fastener head 84 may comprise a drive coupler 104 which can be used to engage the fastener shank 82 with the vertebral body. The drive coupler 104 may include a recess suitable for engaging an Allen-style driver. However, other coupler configurations are also possible.

The fastener 80 may further comprise a flange 86 encircling the head of the fastener 80. In some embodiments, the flange 86 comprises a square shaped protrusion. Other configurations for the flange are also possible. For example, the flange 86 may comprise more than three sides or may comprise a rounded shape. In some embodiments, the flange 86 may only partially encircle the fastener head 84. The flange 86 may advantageously be configured to mate with the inside of the keepers 90, 92, locking the keepers 90, 92 around the fastener head 84. It will be appreciated that the width of the fastener 80 may vary, depending on the dimensions of the other components of the assembly. It will also be appreciated that the length of the fastener 80 may vary. In some embodiments, the dimensions of the fastener 80 may be selected based on properties of the vertebral body in which to fastener 80 is to be inserted (e.g., brittleness, density, strength, etc.).

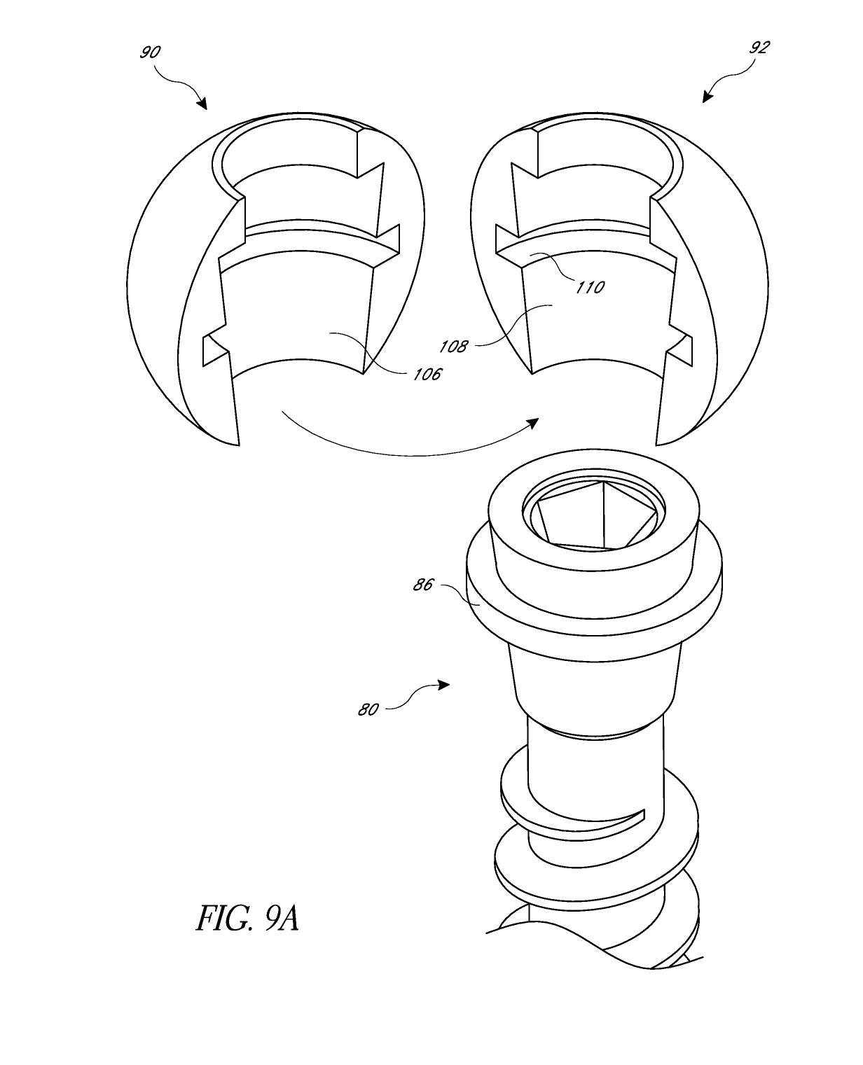

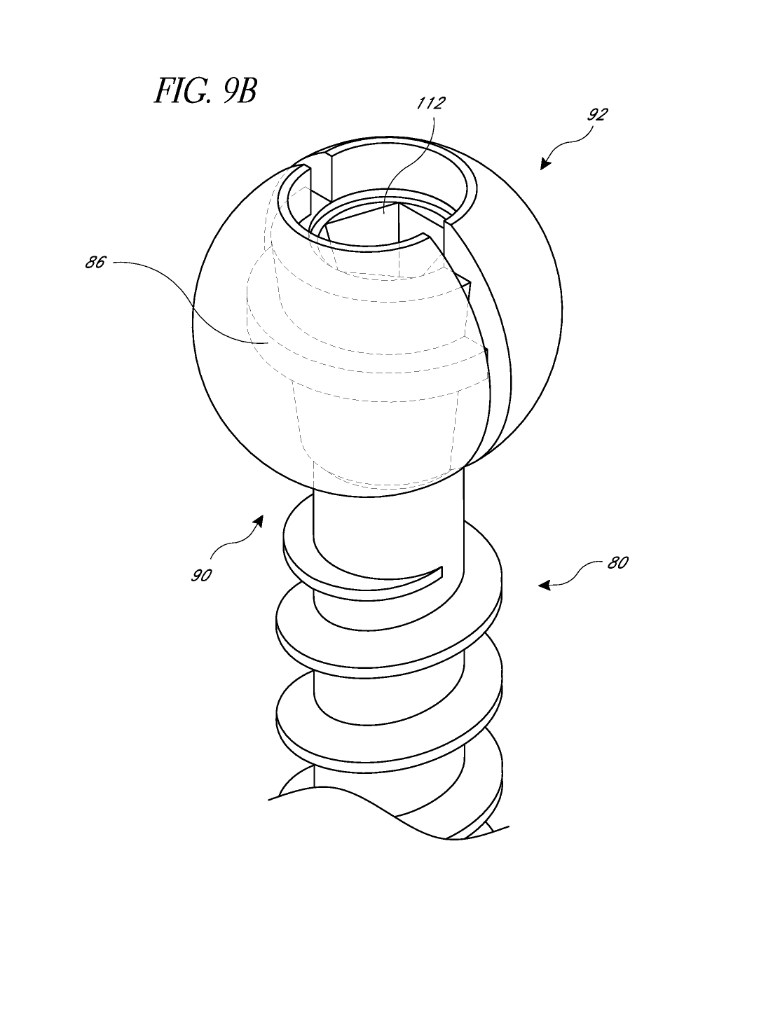

Moving to FIGS. 9A and 913, embodiments of the keeper portions 90, 92 are depicted. As shown in FIG. 9A, the inside surfaces 106, 108 of the keepers 90, 92 are shaped to mate with the head 84 of the fastener 80. The keepers 90, 92 may comprise a square shaped groove 110 configured to mate with the flange 86 of the fastener 80. Other shapes for the groove 110 are also possible. For example, the groove 110 may be rounded or triangular shaped. The mating between the inside surfaces 106, 108 of the keepers 90, 92 to the fastener head 84 and flange 86 may advantageously create a firm hold between the fastener 80 and keepers 90, 92 before they are compressed within the cup 88.

As depicted in FIG. 9B, the keepers 90, 92 may be rounded and form a generally round or spherical shape when surrounding a fastener head. Further, the keepers 90, k may create an aperture 112 above the fastener head 84 which can be used to access the drive coupler 104 of the fastener 80. The keepers 90, 92 and fastener 80 may comprise materials like those described with respect to the keepers and fastener of FIGS. 2-3B.

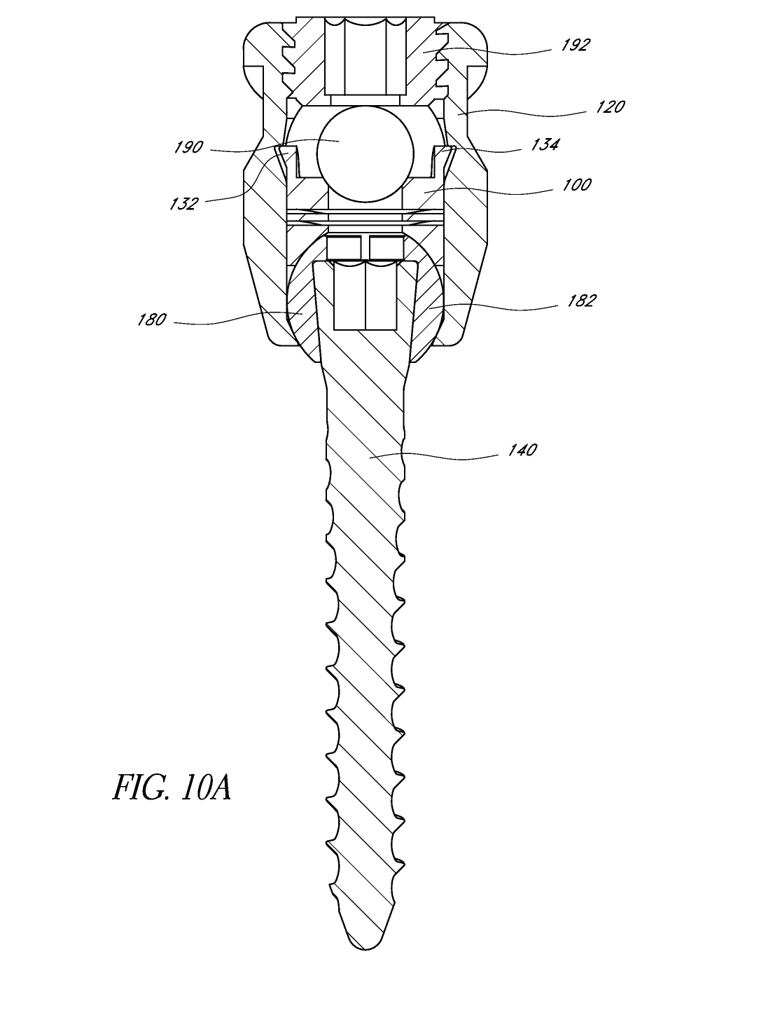

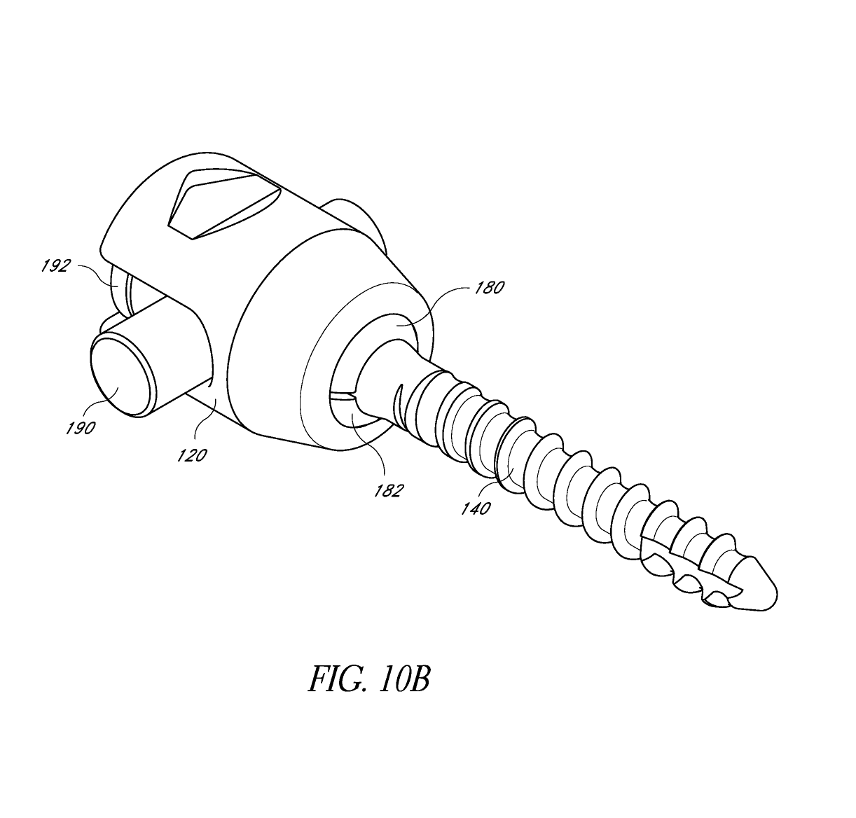

Referring now to FIGS. 10A and 10B, another embodiment of a spinal stabilization assembly is depicted. Unless otherwise described, the components of the assembly in FIGS. 10A and 10B may be similar to the components described with respect to the assembly shown in FIGS. 1A and 1B. FIG. 10A depicts a cross-sectional view of the assembly. The spinal stabilization assembly comprises a fastener 140 extending through a bottom aperture of a cup 120. The fastener 140 comprises an elongated threaded shank and a head. The assembly may also include two keeper portions 180, 182 surrounding the head of the fastener 140. In some embodiments, a compliant saddle 100 may be positioned above the keepers 180, 182. The assembly may further comprise a rod 190 which sits inside the cup 120, above the saddle 100. A threaded compression member 192 may be screwed onto the rod 190, compressing components of the assembly within the cup 120. FIG. 10B depicts a perspective view of the assembly. The bottom of the keepers 180, 182 are visible through a bottom aperture of the cup 120. The rod 190 is shown extending from rod channels of the cup 120 and the compression member 92 is visible through a channel of the cup 120.

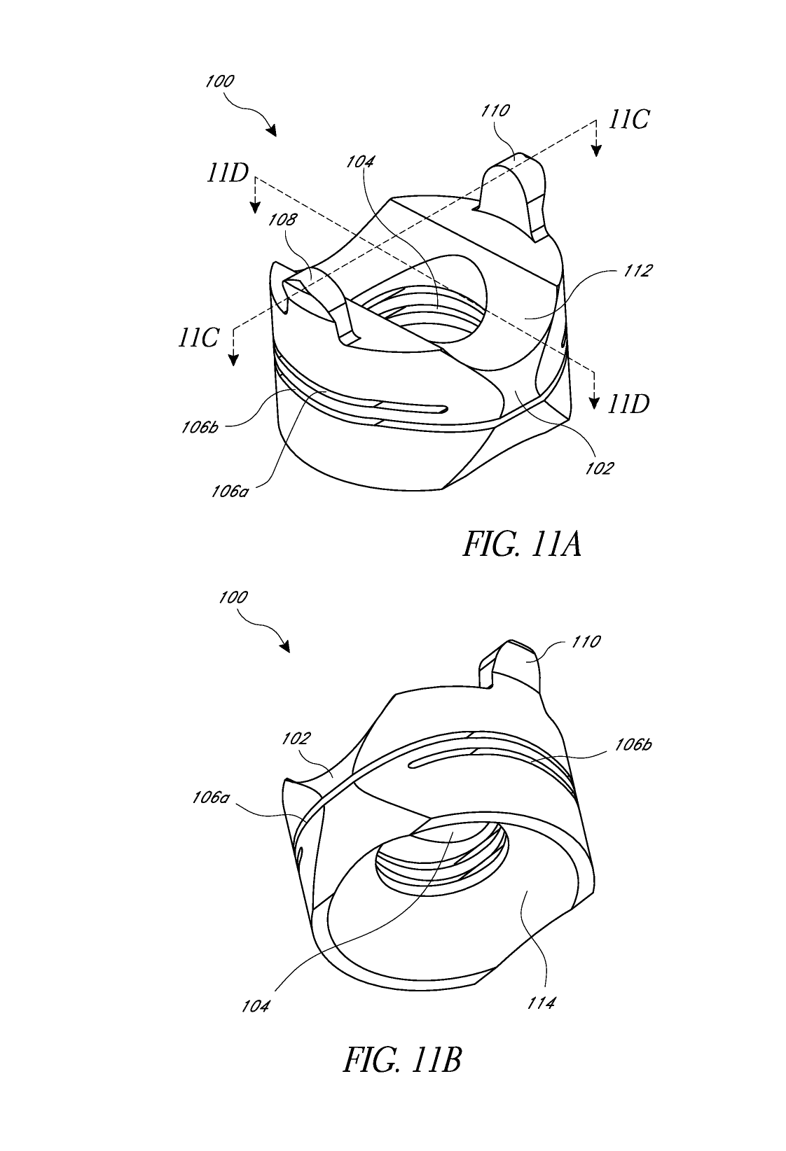

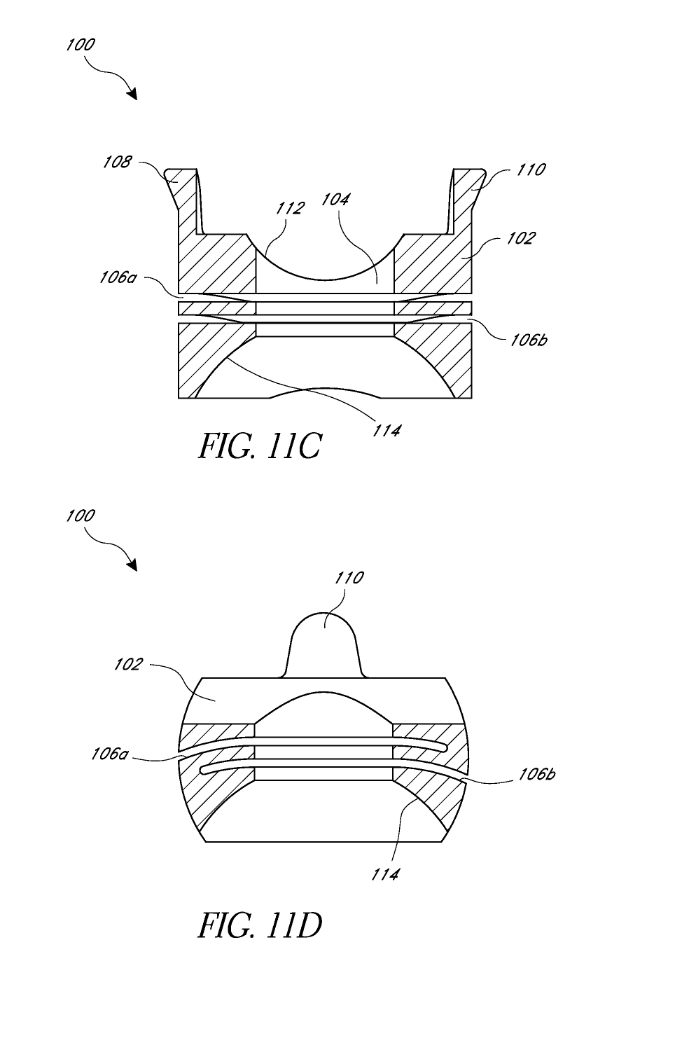

Turning to FIGS. 11A-D, an embodiment of a compliant saddle 100 is depicted. FIGS. 11A and 11B depict elevated views of the saddle and FIGS. 11C and 11D depict cross-sectional views of the saddle 100. The saddle 100 may comprise a body 102 and a center bore 104 to allow access to the drive coupler of a fastener. In some embodiments, the outer surface of the body 102 of the saddle 100 forms a generally cylindrical shape which fits inside the similarly shaped portion of the cup. The upper surface 112 of the saddle 100 may include a surface shaped to mate with a support rod. The bottom surface 114 of the saddle 100 may include a surface shaped to mate with the top of the keepers 180, 182. Other topographies for the top and bottom surfaces 112, 114 of the saddle 100 are also possible.

In some embodiments, the saddle 100 may be compliant to produce spring-like functionality. For example, the saddle 100 of FIGS. 11A-11D comprises slits 106a, 106b on either side of the body 102 of the saddle 100. The slit 106a extends from one side of the outer surface of the body 102 to a point or area part way through the body. The slit 106b extends from the other side of the outer surface of the body 102 and to a point or area part way through the body 102 such that the slit 106b is oriented parallel to and located above the slit 106a. The slits 106a, 106b cut into the saddle 100 may advantageously provide spring-like functionality to the saddle 100. Other configurations for the slits 106a, 106b are also possible. For example in some embodiments, the saddle 100 comprises one or more slits, spaced around the saddle 100 and extending from the outer surface of the body 102 to the center bore 104 of the saddle 100. For another example, the slit or slits may extend from the bore 104 to a point within the body 102. In some embodiments, the slit or slits may extend from the outer surface of the body 102 to a point or area different from that shown in FIGS. 11A-11D. In some embodiments, the slit or slits comprise a more complex configuration. For example, the saddle 100 may comprise a slit in the body 102 of the saddle 100, extending from the outer surface of the body 102 to the center bore 104 of the saddle 100. The slit may continue along the circumference of the saddle 100 while also travelling along the height of the saddle 100, causing the slit to resemble a spiral shape. It will be appreciated that the term slit 106a, 106b encompasses other shapes of portions cut out from the body 102 of the saddle 100. Other mechanisms for imparting spring-like functionality to the saddle 100 are also possible. For example, the saddle 100 may comprise a metal spring that joins separate top and bottom portions of the saddle 100. For another example, the saddle 100 may comprise a deformable material that exerts positive pressure when compressed. Other mechanisms are also possible. It will be appreciated that the term compliant saddle 100 refers to any compressible structure configured to exert pressure on surrounding components when under compression, and is not intended to be limiting by way of requiring a particular structure, feature, or other functionality.

The saddle 100 may also comprise a retaining feature which allows the saddle 100 to remain positioned in a desired orientation within the cup. For example, the saddle 100 of FIGS. 11A and 11B comprises two retaining tabs 108, 110 projecting from the outer surface of the saddle 100. The retaining tabs 108 may be shaped to mate with recesses or depressions 132, 134 on the inner surface of the cup. Other mechanisms for retaining the saddle 100 in a desired orientation (e.g., desired position within the cup, desired amount of compression, etc.) are also possible. For example the recesses and dimple/punch mechanism described with respect to FIGS. 5A and 5B may be used. For another example, a rim within the cup may be used to hold the saddle 100 in place. The retaining feature of the saddle 100 and cup may advantageously utilize the spring-like functionality of the saddle 100 by causing the saddle 100 to sit in a compressed position within the assembly (e.g., as depicted in FIG. 10A). For example the depression or other retaining feature of the cup may be positioned such that the height allotted for the saddle 100 within the cup is less than the height of the saddle 100 in the uncompressed state (e.g., as shown in FIGS. 11A and 11B). In such embodiments, the saddle 100 is compressed and uses at least a portion of the spring travel of the saddle 100. In some embodiments, the saddle 100 uses between about 40% and about 60% of its spring travel. In some embodiments, the saddle 100 uses between about 45% and about 55% of its spring travel. In some embodiments, the saddle 100 uses about 50% of its spring travel. Other amounts are also possible. The downward force exerted by the spring saddle 100 helps compress the components of the assembly, such as the keepers 180, 182 and causes increased friction between the keepers 180, 182 and the cup 120. This extra friction may allow the surgeon to, before placement of the rod 190, orient the cup 120 at a position that will be maintained by the friction between the keepers 180, 182 and the cup 120 as the rod 190 is manipulated into place in the rod channels 126, 128 of the cup 120.

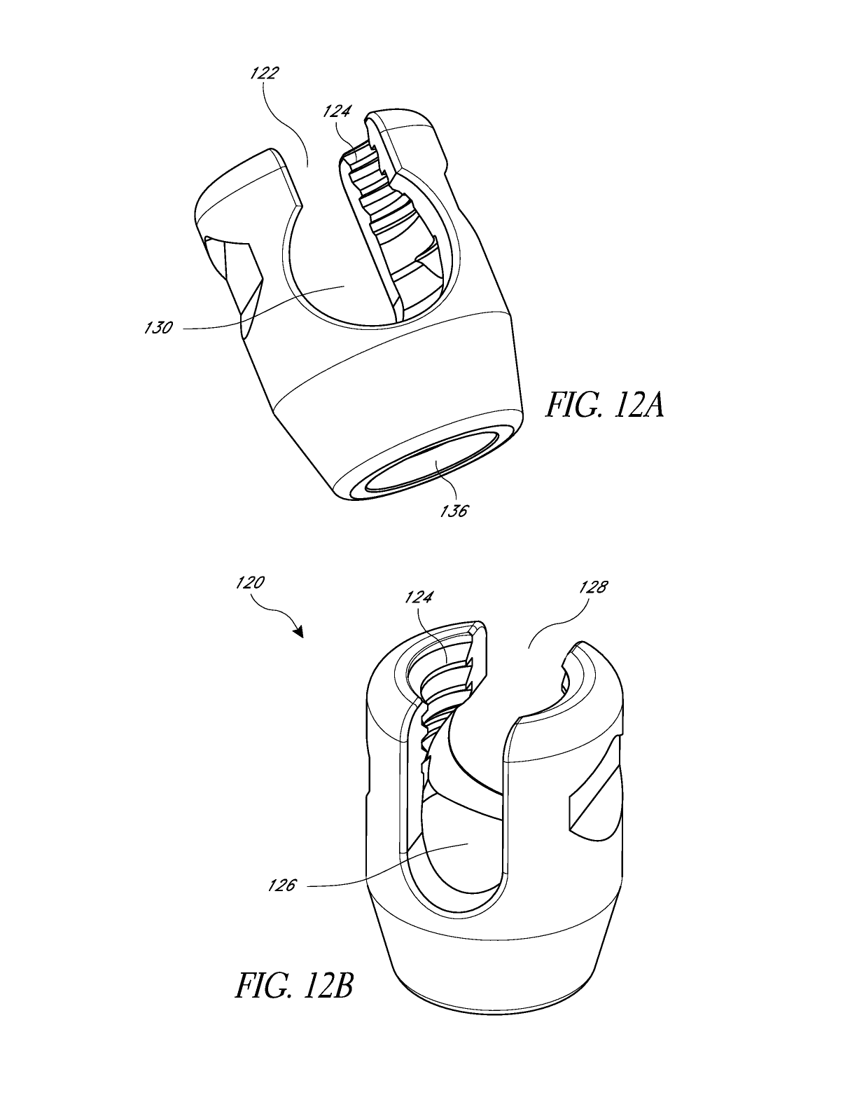

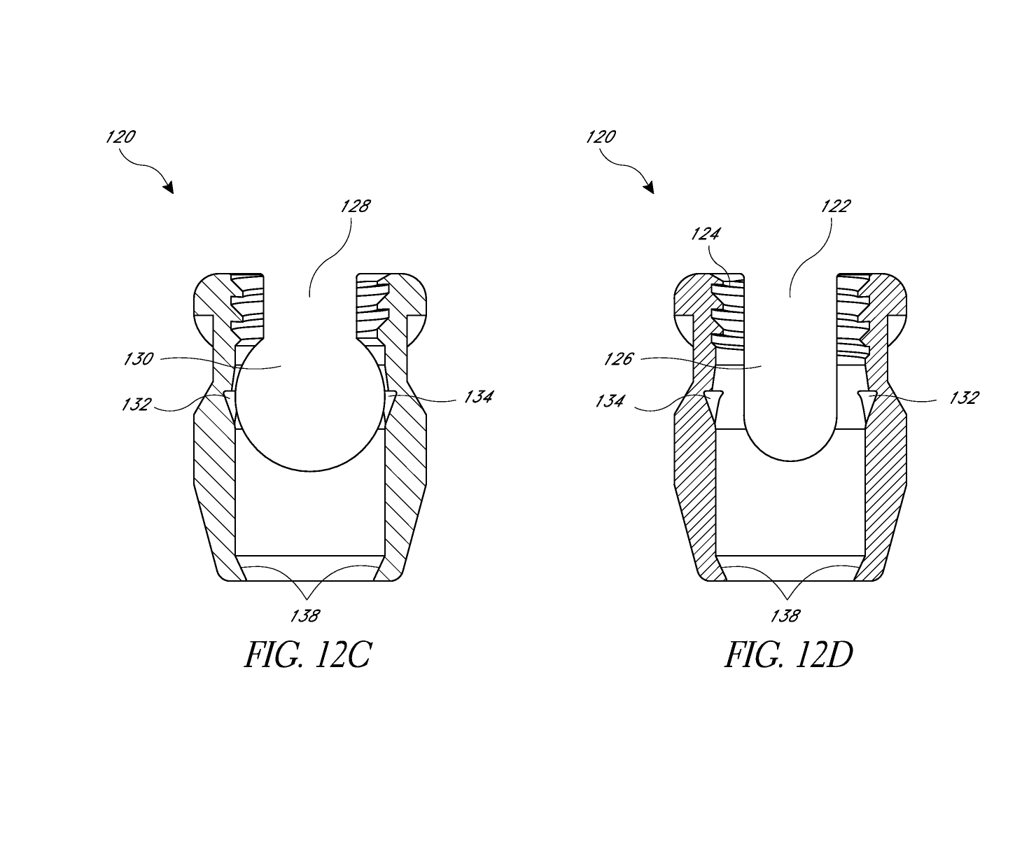

Turning to FIGS. 12A-12D, different perspectives of an embodiment of a cup 120 are shown. FIGS. 12A and 12B depict perspective views of the cup 120 while FIGS. 12C and 12D depict cross-sectional views of the cup 120. As shown in FIGS. 12A and 12D, the cup 120 comprises an upper aperture 122. This aperture 122 may be used for receiving a compression member. In some embodiments, the cup 120 comprises threads 124 located inside the cup 120 and beneath the upper aperture 122 that can be used to engage a threaded compression member. Alternatively, the threads 124 could be located on the outside of the cup 120 to engage a compression member that comprises threads along an inner surface. Other methods for coupling the compression member to the cup 120 are also possible.

The sidewalls of the cup 120 may contain channels 126, 128 (e.g., U-shaped channels), shown in FIGS. 12B-12D, which may be used to receive a rod. FIGS. 12A and 12C show an opening 130 in a sidewall of the cup 120 that can be used for inserting the keeper and saddle 100 components in a manner similar to that described above. The opening 130 may be circular, rectangular, or ovular. Other shapes are also possible.

As depicted in FIGS. 12C and 12D, the inner surface of the sidewalls of the cup 120 may comprise recesses or depressions 132, 134 shaped to mate with the retaining tabs 108, 110 of the saddle 100. The recesses 132, 134 are configured to catch the retaining tabs 108, 110 of the saddle 100 to hold the saddle 100 at a desired orientation (e.g., a desired position, a desired state of compression, etc.) within the cup 120. As described herein, other mechanisms for retaining the saddle 100 at a desired orientation within the cup 120 are also possible. For example the recesses and dimple/punch mechanism described with respect to FIGS. 5A and 5B may be used. For another example, a rim within the cup 120 may be used to hold the saddle in place.

As shown in FIG. 12A, the cup 120 comprises a bottom aperture 136. The bottom aperture 136 may comprise a circumference smaller than the circumference of the keeper portions oriented around the fastener head so that inner surface of the cup 120 around the bottom aperture 136 may support the keepers and the fastener. The shank of the fastener may extend through this aperture as shown in FIGS. 10A and 10B. As shown in FIGS. 12C and 12D, the bottom inner surface 138 of the cup 120 may be angled inwards in a flat conical shape, towards the center of the cup 120, in contrast to the spherical bottom surface 34 of the cup 8 shown in FIGS. 4C and 4D. The flat conical shape may move the contact ring between the cup 120 and the keepers 180, 182 further up along the keepers 180, 182. This higher position of the contact point may advantageously apply more pressure onto the taper of the head of the fastener which may provide a better torsional yield. Further, the flat conical shape of the bottom surface 138 of the cup 120 may provide a larger contact ring than the more rounded bottom surface 34 of the cup 8 shown in FIGS. 4C and 4D. This larger contact ring may provide better compressive bending holding power.

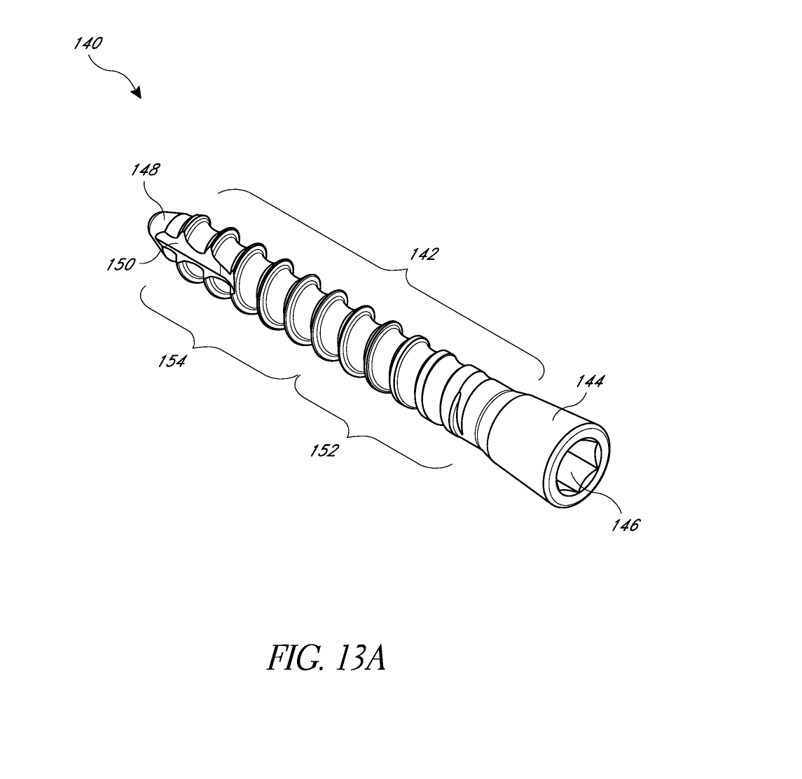

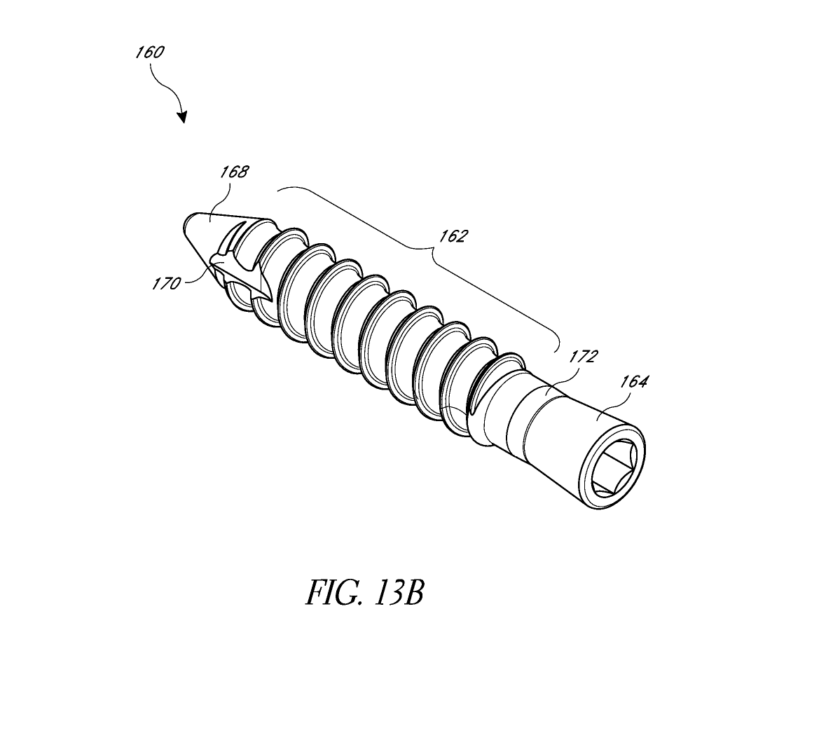

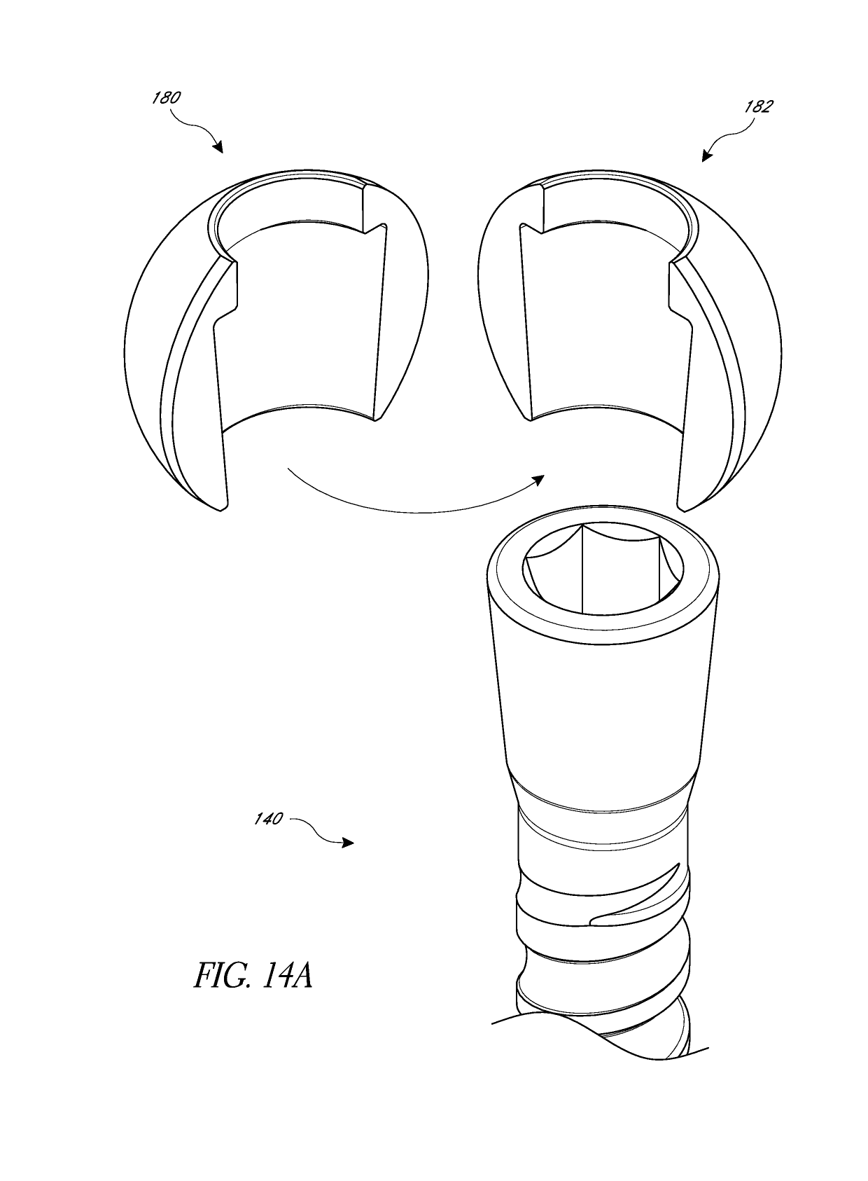

FIGS. 13A-13B illustrate various embodiments of fasteners. FIG. 13A depicts a perspective view of an embodiment of a fastener 140 comprising a threaded shank 142 and a head 144. The head 144 may be frustoconically shaped such that it comprises a tapered cylinder in which the circumference of the top of the head 144 is greater than that of the bottom of the head 144. Other shapes for the head are also possible (e.g., spherical, cylindrical, etc.). The head 144 of the fastener 140 may comprise a drive coupler 146 which can engage a tool used to manipulate the fastener 140 in order to threadably engage the shank 142 of the fastener 140 with the vertebral body. The shank 142 may include a tip 148 suitable for piercing bone such as a pedicle. The threaded shank 142 may also comprise a groove 150 located near the tip 148 of the shank 142. This groove 150 can create a self-tapping screw as the edges of the groove 150 are capable of cutting the bone to create a thread pattern. The groove 150 may be rounded, angular, or square. Other configurations are also possible. In some embodiments, the fastener 140 is not capable of piercing or threading the bone. In such embodiments a hole, either tapped or untapped, may be drilled for the fastener 140.

FIG. 13B illustrates another embodiment of a fastener 160 comprising a threaded shank 162 and a head 164. The head 164 may be frustoconically shaped such that it comprises a tapered cylinder in which the circumference of the top of the head 164 is greater than that of the bottom of the head 164. Other shapes for the head are also possible (e.g., spherical, cylindrical, etc.). The head 164 may comprise a drive coupler 166 which can engage a tool used to manipulate the fastener 160 in order to threadably engage the shank 162 of the fastener 160 with the vertebral body. In some embodiments, the drive coupler 166 comprises a recess whose shape may be one of a variety of forms. For example, the recess may be hexagonal in order to engage an Allen-style driver. However, other shapes and methods of coupling are also possible. The threaded shank 162 may comprise a tip 168 suitable for piercing bone such as a pedicle. The threaded shank 162 may also comprise a groove 170 located near the tip 168 of the shank 162. This groove 170 can crate a self-tapping fastener 160 as the edges of the groove 170 are capable of cutting the bone to create a thread pattern. The groove 170 may be rounded, angular or square. Other configurations are also possible. In some embodiments, the fastener 140 is not capable of piercing or threading the bone. In such embodiments a hole, either tapped or untapped, may be drilled for the fastener 140.

It will be appreciated that the width of the fasteners 140, 160 may vary, depending on the dimensions of the other components of the assembly. It will also be appreciated that the length of the fasteners 140, 160 may vary. In some embodiments, the dimensions of the fastener 140, 160 may be selected based on properties of the vertebral body into which fastener 140, 160 is to be inserted (e.g., brittleness, density, strength, etc.).

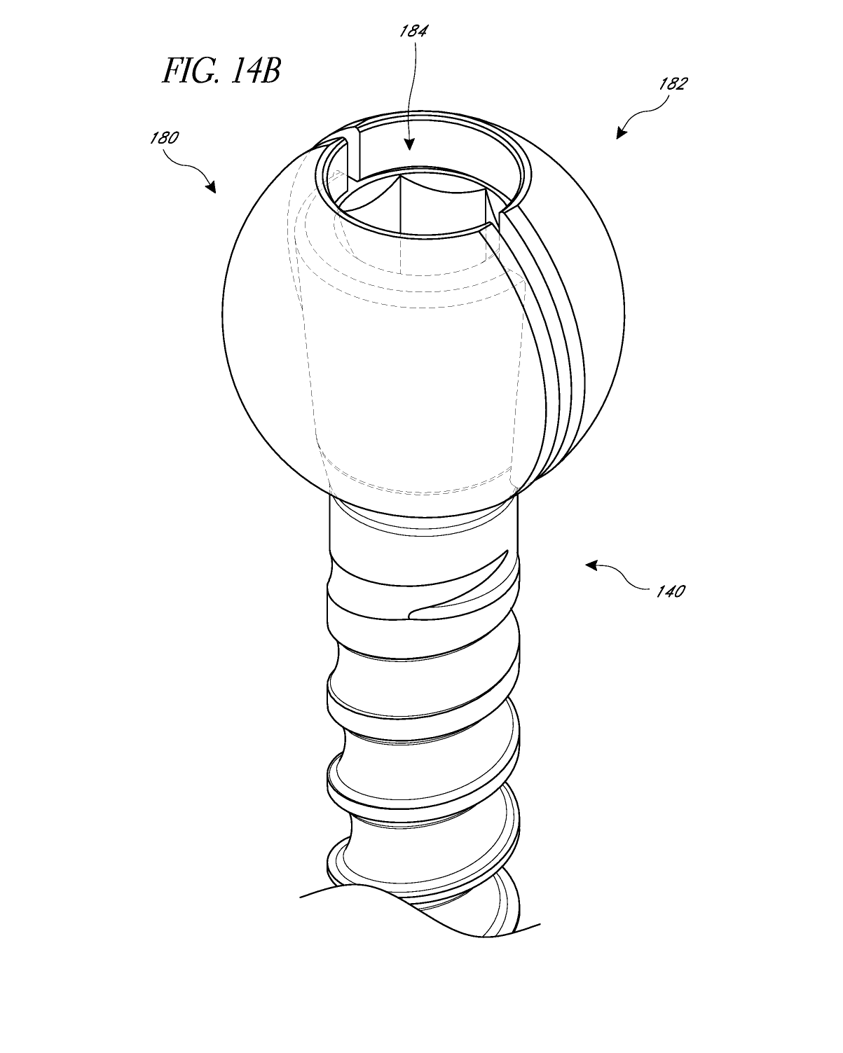

Referring now to FIGS. 14A and 14B, embodiments of keeper portions 180, 182 are depicted. As shown in FIG. 14A, the inside surfaces of the keepers 180, 182 are shaped to mate with the head of a fastener (e.g., fastener 140, fastener 160). The keepers 180, 182, shown surrounding fastener head 144 in FIG. 14B, may be rounded and form a generally round or spherical shape when oriented around a fastener head. Further, the keepers 180, 182 may create an aperture 184 above the fastener head 144 which can be used to access the drive coupler 146 of the fastener 140. Unlike the keepers 4, 6, 90, 92 described with respect to FIGS. 3A and 3B or FIGS. 9A and 9B, the keepers 180, 182 of FIGS. 14A and 14B do not comprise a groove shaped to mate with a flange, a ring, or the like. The positive pressure exerted onto the keepers 180, 182 by the saddle may advantageously be sufficient to compress the keepers 180, 182 around the head 144 of the fastener 140. However, it will be appreciated that the keepers 180, 182 may comprise a groove and the fastener 140 may comprise a flange or ring or the like. The keepers 180, 182 and fasteners 140, 160 may comprise materials like those described with respect to the keepers and fastener of FIGS. 2-3B.

It will be appreciated that spinal stabilization assemblies described herein may comprise any combination of the components described herein. It will also be appreciated that the components described herein may comprise any one of the features described herein, combinations of the same, or the like.

A method of assembling the embodiment of the device shown in FIGS. 1A and 1B may include inserting keeper 6 with ring 78 in its groove 76 through opening 42 in the sidewall of the cup 8. The fastener 2 is then pushed up through the bottom aperture 32 of the cup 8 and through the ring 78. Keeper 4 is then inserted through opening 42 to enclose the head 22 of the fastener 2 with the ring 78 inside the keepers 4, 6. The head 22 of the fastener 2, along with the attached keepers 4, 6, is then pulled down into the seat 34 in the bottom of the cup 8. The saddle 10 is then pushed into the cup 8 through the opening 42 in the sidewall. The recesses 68, 70 of the saddle 10 should be aligned with recesses 48, 50 of the cup 8. A punch or similar tool may be used to punch or dimple the sidewalls of the cup 8 at the recesses 48, 50, deforming the cup 8 into the recesses 68, 70 of the saddle 10, holding the saddle 10 in place.

Assembly of the device of FIGS. 7-9B is somewhat less complex because there is no separate ring to position with the screw head and keepers during assembly. In this embodiment, keeper 92 is inserted through opening 42 in the sidewall of the cup 88. The fastener 80 is then pushed up through the bottom aperture of the cup 88. Keeper 90 is then inserted through opening 42 to enclose the head of the fastener 2. The remaining steps are the same.

Assembly of the device of FIGS. 10A-14B is also less complex than that of the device shown in FIGS. 1A and 1B as there is no separate ring to position with the screw head and keepers during assembly. Furthermore, punching or dimpling of the sidewalls of the cup into the saddle is not required. Like the assembly of the device of FIGS. 7-9B, in this embodiment, keeper 180 is inserted through the opening 130 in the sidewall of the cup 120. The fastener is then pushed up through the bottom aperture 136 of the cup 120. Keeper 182 is then inserted through the opening 130 to enclose the head 144 of the fastener 140. The head 144 of the fastener 140, along with the attached keepers 180, 182, is then pulled down into the seat 138 in the bottom of the cup 120. The saddle 100 is then pushed into the cup 120 through the opening 130 in the sidewall. The retaining tabs of the saddle should be aligned with recesses of the cup. The saddle is pushed down and compressed against the top of the keepers until the retaining tabs catch in the recesses of the cup.

As described above, in the embodiment with the ring 78, the ring 78 is operative to push the keepers outward to cause friction with the inner cup surface for convenient orientation of the cup on the screw head. In the embodiment with the flange 86 attached to the screw head, this function is not performed, but the flange may aid in creating a firm hold between the fastener head and the keepers. In the embodiment with the compliant saddle, the compliant saddle is operative to exert a downward force on the keepers, performing the same function that the ring performs in the embodiment of FIG. 1A. The compliant saddle may be incorporated into the embodiment with the ring 78 or the flange 86 to provide or enhance the coupling or friction between the keepers and the fastener head or the keepers and the cup.

The surgeons receive completed assemblies of cups with screws, keepers, and saddles installed. During surgery, a surgeon will screw the fastener 2 into the bone, with the cup and saddle assembly attached, by extending a driver tip through the upper aperture 44 of the cup 8 and through the openings 60, 31 in the saddle and keepers to access the head 22 of the fastener 2 with the driver tip. This is repeated for two or more screws. The rod 14 is then placed in the rod channels 38, 40 of each cup 8. The surgeon then inserts the compression members 12 through the upper apertures 44 of the cups 8 and tightens them down to lock the components in place with the rod extending between the installed screws.

The foregoing description details certain embodiments of the invention. It will be appreciated, however, that no matter how detailed the foregoing appears in text, the invention can be practiced in many ways. As is also stated above, it should be noted that the use of particular terminology when describing certain features or aspects of the invention should not be taken to imply that the terminology is being re-defined herein to be restricted to including any specific characteristics of the features or aspects of the invention with which that terminology is associated. The scope of the invention should therefore be construed in accordance with the appended claims and any equivalents thereof.

What is claimed is:

1. A spinal stabilization assembly comprising
a cup comprising an aperture at its lower end and opposed rod channels;
a fastener extending through the aperture at the lower end of the cup and comprising a head and a body; and
a plurality of keeper portions, said keeper portions having an outer surface and an inner surface; wherein said inner surface is shaped to mate with said head of said fastener; wherein said keeper portions at least partially surround the head of the fastener; and wherein said keeper portions have at least some of their outer surface in contact with an internal surface of the cup.

2. The spinal stabilization assembly of claim 1, further comprising a saddle located above the fastener.

3. The spinal stabilization assembly of claim 2, wherein the saddle is configured to exert pressure on the plurality of keeper portions.

4. The spinal stabilization assembly of claim 3, wherein a body of the saddle is compliant.

5. The spinal stabilization assembly of claim 1, further comprising a compression member attached at the upper aperture of the cup.

6. The spinal stabilization assembly of claim 5 further comprising a rod extending through the rod channels of the cup and positioned between the compression member and the head of the fastener.

7. The spinal stabilization assembly of claim 5, wherein the compression member comprises threads that mate with a threaded portion of the cup.

8. The spinal stabilization assembly of claim 1, wherein the keeper portions comprise a groove.

9. The spinal stabilization assembly of claim 9, wherein a ring is positioned in the groove.

10. The spinal stabilization assembly of claim 9, wherein a flange at least partially encircling the head of the fastener is positioned in the groove.

11. The spinal stabilization assembly of claim 1, wherein the keeper portions comprise a material softer than the material comprising the head of the fastener.

12. The spinal stabilization assembly of claim 1, wherein the fastener comprises a tapered cylindrical head.

13. The spinal stabilization assembly of claim 1 wherein the fastener comprises a pedicle screw.

14. The spinal stabilization assembly of claim 1, wherein the body of the fastener tapers outwardly in a portion proximate to the head of the fastener.

15. The spinal stabilization assembly of claim 1, wherein the body of the fastener comprises a lip proximate to the head of the fastener.

16. A method for assembling a spinal stabilization assembly comprising
inserting a fastener through an aperture of a cup;
inserting one or more keeper portions through an aperture in a side wall of the cup; and
arranging the one or more keeper portions around a head of the fastener such that the keeper portions at least partially surround the head of the fastener and have their outer surface in contact with an internal surface of the cup

17. The method of claim 16, further comprising inserting a saddle through an aperture of the cup and placing the saddle so that its bottom portion mates with the outer surface of the plurality of keeper portions.

18. The method of claim 17, further comprising compressing the saddle and fixing it in the compressed state.

19. The method of claim 17, further comprising placing a rod on top of an upper portion of the saddle, the rod extending through one or more side channels of the cup.

20. The method of claim 16, further comprising inserting a compression member through an upper aperture of the cup and coupling it to the cup so that it exerts pressure on the fastener.

21. The method of claim 16 wherein the fastener comprises a pedicle screw.

22. A spinal stabilization assembly comprising
a cup comprising an aperture at its lower end and opposed rod channels;
a fastener extending through the aperture at the lower end of the cup and comprising a head and a body; and
means for clamping the head of the fastener into the cup said means positioned between the cup and the head of the fastener.

23. The spinal stabilization assembly of claim 22, further comprising means for biasing the clamping means outward toward the cup.

24. The spinal stabilization assembly of claim 22, wherein the fastener comprises a flange at least partially encircling its head.

25. The spinal stabilization assembly of claim 22, further comprising a compliant saddle.

26. A spinal stabilization assembly comprising
a cup comprising an aperture at its lower end and opposed rod channels;
a fastener extending through the aperture at the lower end of the cup and comprising a head and a body; and
one or more keeper portions, said one or more keeper portions having an outer surface and an inner surface; wherein said inner surface is shaped to mate with said head of said fastener; wherein said one or more keeper portions at least partially surrounds the head of the fastener; wherein said one or more keeper portions has at least some of its outer surface in contact with an internal surface of the cup; and wherein said one or more keeper portions comprises a material that is softer than the material comprising said fastener or said cup.

27. The spinal stabilization assembly of claim 26, wherein the one or more keeper portions comprise a material with a yield strength of less than or equal to about 50 ksi.

28. The spinal stabilization assembly of claim 26, wherein one or both of the fastener and the cup comprise a material with a yield strength of greater than or equal to about 100 ksi.

29. A spinal stabilization assembly comprising
a cup comprising an aperture at its lower end and opposed rod channels;
a fastener extending through the aperture at the lower end of the cup and comprising a head and a body; and
a compliant saddle positioned above the fastener.

30. The spinal stabilization assembly of claim 29, comprising a plurality of keeper portions, said keeper portions having an outer surface and an inner surface; wherein said inner surface is shaped to mate with said head of said fastener; wherein said keeper portions at least partially surround the head of the fastener; and wherein said keeper portions have at least some of their outer surface in contact with an internal surface of the cup.
